(19) United States
(12) Patent Application Publication
(10) Pub. No.: US 2026/0362600 A1
(43) Pub. Date: Nov. 12, 2026

(54) CIRCUIT BOARD AND ULTRASONIC DIAGNOSTIC APPARATUS

(71) Applicant: Konica Minolta, Inc., Tokyo (JP)

(72) Inventor: Kazuya Osada, Tokyo (JP)

(73) Assignee: Konica Minolta, Inc., Tokyo (JP)

(21) Appl. No.: 19/672,096

(22) Filed: May 8, 2026

(30) Foreign Application Priority Data
May 12, 2025 (JP) 2025-079612

(51) Int. Cl. H05K 1/18; A61B 8/00

(52) U.S. Cl. CPC H05K 1/18; A61B 8/4477; H05K 2201/10015; H05K 2201/10053

(57) ABSTRACT

A circuit board includes, a connector to be connected to a main body of an ultrasonic diagnostic apparatus; a first connection circuit including a first mount on which a connection terminal used for connection to an ultrasound probe is to be mounted, and a second mount located between the first mount and the connector and on which a switching circuit is to be mounted; and a second connection circuit including a third mount on which a passive component is to be mounted. Either the connection terminal is mounted on all of the first mounts, the switching circuit is mounted on all of the second mounts, and the passive component is not mounted on any third mount, or the switching circuit is not mounted on at least one of the second mounts, and the passive component is mounted on at least one of the third mount.

### CROSS-REFERENCE TO RELATED APPLICATION

The entire disclosure of Japanese Patent Application No. 2025-079612, filed on May 12, 2025, including description, claims, drawings and abstract is incorporated herein by reference.

### BACKGROUND OF THE INVENTION

### Technical Field

The present disclosure relates to a circuit board and an ultrasonic diagnostic apparatus.

### Description of Related Art

An ultrasonic diagnostic apparatus for non-invasively measuring an interior of a living body or an object is provided with an ultrasound probe, i.e., a probe, and controls transmission and reception of ultrasound by the ultrasound probe. There are a plurality of types of ultrasound probes each capable of transmitting and receiving ultrasound in an appropriate direction or scanning pattern depending on a diagnostic target. To reduce the time and effort required to replace the probe, the ultrasonic diagnostic apparatus may include a plurality of connection terminals such that a plurality of probes can be connected in parallel. Japanese Unexamined Patent Application Publication No. 2010-46120 discloses an ultrasonic diagnostic apparatus that can be provided with any number of connection ports by adding an expansion unit.

Although a number of probes required varies depending on the user, preparing respective circuit boards for ultrasonic diagnostic apparatuses having different numbers of connection terminals is time-consuming and costly. However, since an ultrasound probe has a large number of channels, the impedance of the entire circuit varies significantly depending on whether a signal line of each channel is connected to a main body of the ultrasonic diagnostic apparatus. Therefore, merely changing a number of connection terminals on a common circuit board adversely affects the signal, making it impossible to perform highly accurate measurements.

### SUMMARY OF THE INVENTION

An object of the present disclosure is to provide a circuit board and an ultrasonic diagnostic apparatus capable of connecting an appropriate number of ultrasound probes while suppressing a decrease in measurement accuracy.

To achieve the above object, according to one aspect of the present disclosure, the circuit board reflecting one aspect of the present disclosure includes:
a connector to be connected to a main body of an ultrasonic diagnostic apparatus;
a first connection circuit including a first mount which includes one end at the connector, and on which a connection terminal used for connection to an ultrasound probe is to be mounted, and a second mount located between the first mount and the connector and on which a switching circuit is to be mounted; and
a second connection circuit including a third mount which includes one end at the connector, and on which a passive component is to be mounted,
wherein,
in at least one first connection circuit, the connection terminal is mounted on the first mount, and the switching circuit is mounted on the second mount,
at least two sets of the first mount and the second mount are provided, and
either the connection terminal is mounted on all of the first mounts, the switching circuit is mounted on all of the second mounts, and the passive component is not mounted on any third mount, or the switching circuit is not mounted on at least one of the second mounts, and the passive component is mounted on at least one of the third mount.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a block diagram illustrating a functional configuration of an ultrasonic diagnostic apparatus including a circuit board;

FIG. 2 illustrates another example of the circuit board;

FIG. 3 illustrates another example of the circuit board;

FIG. 4 illustrates another example of the circuit board;

FIG. 5 illustrates another example of the circuit board;

FIG. 6 illustrates another example of the circuit board; and

FIG. 7 illustrates another example of the circuit board.

### DETAILED DESCRIPTION

Hereinafter, one or more embodiments of the present disclosure will be described with reference to the drawings. However, the scope of the invention is not limited to the disclosed embodiments.

Hereinafter, embodiments of the present invention will be described with reference to the drawings.

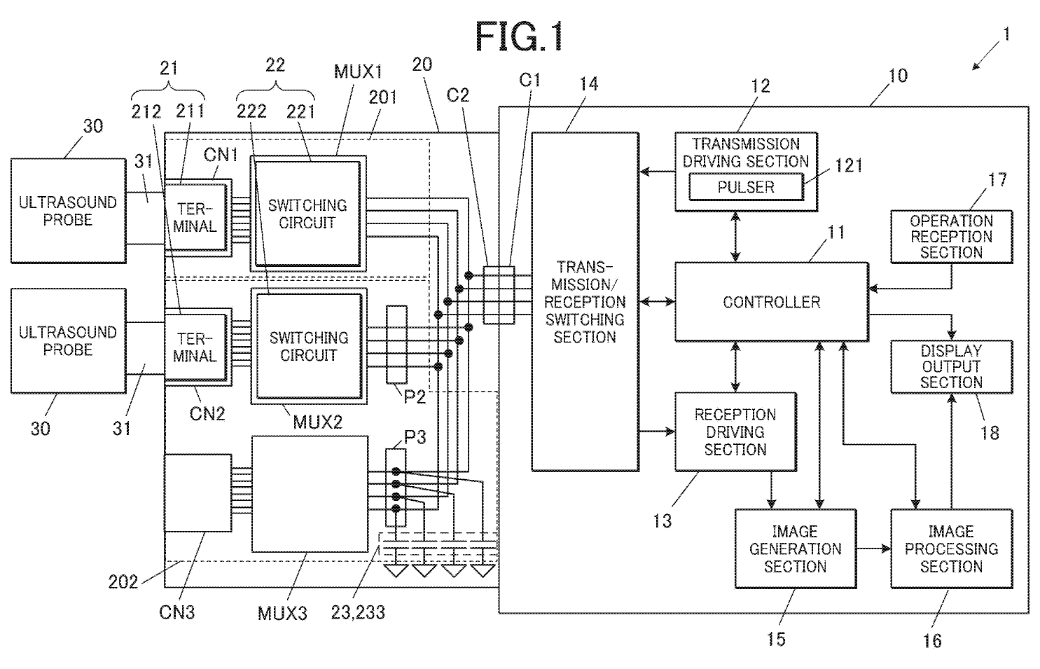

FIG. 1 is a block diagram illustrating a functional configuration of an ultrasonic diagnostic apparatus 1 including a circuit board 20 according to the present embodiment. The ultrasonic diagnostic apparatus 1 includes a main body 10, the circuit board 20, and an ultrasound probe 30.

The ultrasound probe 30 transmits and receives ultrasound at a drive frequency of about 1 to 30 MHz to measure an interior of a subject such as a living body. The ultrasound probe 30 includes a plurality of transducers and a cable 31. Each transducer may be a piezoelectric element configured to deform in response to an electrical signal. The transducer transmits ultrasound in accordance with deformation. Each transducer is deformed in response to a reflected wave (i.e., an echo) of transmitted ultrasound and outputs the electrical signal, thereby functioning as an acoustic sensor that detects the reflected wave.

The cable 31 includes, at one end thereof, a connector configured for connection to the circuit board 20. The transducers and the connector are connected in parallel via a plurality of signal lines equal in number to the transducers. The connector is removably attachable to the circuit board 20. The user brings an ultrasound transmission/reception surface of the ultrasound probe 30, i.e., a surface facing a direction in which ultrasound is emitted from the transducer, into contact with the subject with an appropriate pressure, and operates the ultrasonic diagnostic apparatus 1 to perform ultrasound diagnosis.

The plurality of transducers may be arranged in a one-dimensional array aligned in a predetermined scanning direction, or may be arranged in a two-dimensional array. The number of transducers may be, for example, about 32 to 256. When voltage pulses are sequentially supplied to the transducers, each piezoelectric material deforms in accordance with an electric field generated in the piezoelectric material, thereby emitting ultrasound. By synthesizing ultrasound in accordance with differences in oscillation timing among the transducers, directivity can be imparted to an ultrasound emission direction. A suitable directivity is generally determined in accordance with examination content, and, depending on the directivity, the ultrasound probe 30 may be of a linear scanning type, a sector scanning type, a convex scanning type, and the like. The scanning may be an electronic scanning method or a mechanical scanning method.

The main body 10 is the main body of the ultrasonic diagnostic apparatus 1, and performs analysis processing and display of measurement results based on measurement data acquired by the ultrasound probe 30. The main body 10 includes a controller 11, a transmission driving section 12, a reception driving section 13, a transmission/reception switching section 14, a connection terminal C1, an image generation section 15, an image processing section 16, an operation reception section 17, a display output section 18, and the like.

The controller 11 is capable of operating based on signals from input devices, such as an input key of the operation reception section 17 or a pointing device, in response to an external input operation. The controller 11 includes a central processing unit (CPU), a random access memory (RAM), and a nonvolatile storage unit, among other components. The CPU performs arithmetic processing to control the overall operation of the ultrasonic diagnostic apparatus 1. The CPU may be a single processor, or a plurality of processors may perform computations in parallel or independently of each other depending on the application or the like. The CPU may be a general-purpose processor or a dedicated processor suitably designed for arithmetic processing related to the ultrasound diagnosis.

The RAM provides a working memory space for the CPU and stores temporary data. The temporary data may include measurement data obtained by the ultrasound probe 30, however, a RAM for storing the measurement data may be provided separately in the image generation section or the image processing section. The RAM may be DRAM, SRAM, or any other type of memory usable as RAM.

The nonvolatile storage section stores programs, setting data, and the like. The nonvolatile storage section may include an HDD (Hard Disk Drive), an SSD (Solid State Drive), or the like. The storage section may include a flash memory of the like.

Part of or the entire storage section may be an externally attached peripheral device and/or may be located on a network. Part of or the entire storage section may be a cloud server or the like. The programs include an operation control program for generating electrical pulses to cause the ultrasound probe 30 to transmit ultrasound, and a control program for processing and analyzing echo signals of the ultrasound received by the ultrasound probe 30. In addition, the programs may include a program for causing the display output section 18 to display an analysis result, a setting program for switching an output pattern of the ultrasound, and the like.

The transmission driving section 12 generates and outputs a pulse signal to be supplied to the ultrasound probe 30 in accordance with a control signal input from the controller 11, thereby causing the ultrasound probe 30 to generate ultrasound. The transmission driving section 12 may include, for example, a clock generation circuit, a pulser 121, a pulse width setting section, and a delay circuit. The clock generation circuit is a circuit that generates a clock signal that determines transmission timing and transmission frequency of the pulse signal. The pulse width setting section sets a waveform, a voltage amplitude, and a pulse width of a transmission pulse to be output from the pulser 121. The pulser 121 generates a transmission pulse based on a pulse width setting, and outputs the transmission pulse to a wiring path corresponding to each transducer of the ultrasound probe 30. The delay circuit counts the clock signal output from the clock generation circuit and, when a set delay time has elapsed, causes the pulser 121 to generate the transmission pulse and output the transmission pulse to each wiring path.

The reception driving section 13 is a circuit that acquires a reception signal input from the ultrasound probe 30 under control of the controller 11. The reception driving section 13 may include, for example, an amplifier, an A/D conversion circuit, and a phasing addition circuit. The amplifier is a circuit that amplifies, at a preset gain, the reception signal corresponding to ultrasound received by respective transducers of the ultrasound probe 30. The A/D conversion circuit is a circuit that converts the amplified reception signal into digital data at a predetermined sampling frequency. The phasing addition circuit applies a delay time to the A/D-converted reception signal for each wiring path corresponding to each transducer to align phases, and generates sound ray data by summing the phase-aligned signals.

Based on control by the controller 11, the transmission/reception switching section 14 switches a connection with the circuit board 20 between the transmission driving section 12 and the reception driving section 13 depending on whether the transducer emits ultrasound or receives ultrasound. The switching operation may be performed automatically and periodically in accordance with an expected time at which an ultrasound echo returns.

The connection terminal C1 is a terminal that connects the transmission/reception switching section 14 to the circuit board 20. The terminal may be set as appropriate in accordance with, for example, the number of wiring paths that are provided.

The image generation section 15 generates a diagnostic ultrasound image based on ultrasound reception data. The image generation section 15 detects the sound ray data input from the reception driving section 13 to obtain a signal. The detection may be envelope detection. In addition, as necessary, the image generation section 15 performs one or a combination of logarithmic amplification, filtering such as low-pass filtering or smoothing, and enhancement processing. The image generation section 15 is capable of performing dynamic range adjustment for display, gamma correction, and the like.

The image processing section 16 performs processing for detecting a specific detection target or the like from the generated ultrasound image and performing highlighted display or the like. The image processing section 16 may include a machine learning model for image processing. The image processing section 16 may calculate differences among a plurality of items of image data, or may convert the image data between a spatial axis and a frequency axis by, for example, a Fourier transform.

The image generation section 15 and/or the image processing section 16 may include a dedicated CPU and/or RAM used for image generation, separately from the CPU and the RAM of the controller 11. The dedicated hardware configuration for image generation may be formed on a substrate (e.g., a board) as, for example, an application-specific integrated circuit (ASIC).

Alternatively, the image generation section 15 and/or the image processing section 16 may have no dedicated hardware configuration, and processing may be performed by the CPU and the RAM of the controller 11.

The operation reception section 17 includes a push-button switch, an input key, a pointing device, and the like, receives a user input operation, converts the user input operation into the electrical signal, and outputs the electrical signal to the controller 11. The input key may be a keyboard. The pointing device may be a mouse, a trackball, a touch sensor on a display screen, or a combination thereof.

The display output section 18 includes a display screen that uses any of various display methods, and a drive section therefor. Examples of the various display methods include a liquid crystal display (LCD), an organic electroluminescent (EL) display, an inorganic EL display, a plasma display, and the like.

However, the display method is not limited thereto. The display output section 18 generates, under control of the controller 11, a drive signal for the display screen, and displays on the display screen a menu and status related to the ultrasound diagnosis and the measurement data based on the received ultrasound. The display content may include the image data generated by the image processing section 16. In addition, the display output section 18 may further include an LED lamp or the like to indicate whether power is on or off.

The operation reception section 17 and the display output section 18 may be integrally configured with the main body 10. Alternatively, the operation reception section 17 and the display output section 18 may each be a detachable peripheral device connectable via an RGB cable, a USB cable, an HDMI (registered trademark) cable, or the like.

Among the components of the main body 10, at least a portion including the transmission driving section 12 and the connection terminal C1 may be located on the substrate. The substrate may be stacked with the circuit board 20, and the connection terminal C1 and a connection terminal (connector) C2 may be connected to each other.

The circuit board 20 includes circuitry for connecting the main body 10 and the ultrasound probe 30 to transmit and receive data. The circuit board 20 includes a maximum number of mounts of connection circuits to enable connection of the ultrasound probe 30. The maximum number of mounts is at least two, and may be three, for example. However, the circuit board 20 may have a configuration capable of connecting the number of ultrasound probes 30 that is fewer than the maximum number of mounts. For example, the circuit board 20 may be configured to be able to connect up to two ultrasound probes 30 as a maximum connectable number. When a plurality of ultrasound probes 30 are connectable, which connection circuit is to be used can be switched and set via the operation reception section 17 of the main body 10. That is, even when a plurality of ultrasound probes 30 are simultaneously connected, the main body 10 can selectively transmit and receive data to and from the ultrasound probe 30 connected to a designated connection circuit.

The circuit board 20 may include a first connection circuit 201, and second connection circuits 202 and 203. The sum of the number of the first connection circuits 201 and the number of the second connection circuits 202 and 203 is the maximum number of mounts. In one embodiment, the number of the first connection circuit may be one, and there may be a plurality of second connection circuits. One end of each of the first connection circuit 201 and the second connection circuits 202 and 203 is the connection terminal C2. That is, the first connection circuit 201, and the second connection circuits 202 and 203 extend in parallel from the connection terminal C2. Note that the first connection circuit 201 and the second connection circuits 202 and 203 may be connected to the connection terminal C2 via a common wiring line formed by merging. The connection terminal C2 as the connecting section may have a terminal structure that is directly connectable to the connection terminal C1 of the main body 10.

The first connection circuit 201 includes a first mount CN1 for mounting a terminal 211 that is the connection terminal to be connected to the ultrasound probe 30, and a second mount MUX1 for mounting a switching circuit 221. The second connection circuit 202 includes a first mount CN2, a second mount MUX2, and a third mount P2 for mounting a passive component 23. The second connection circuit 203 includes a first mount CN3, a second mount MUX3, and a third mount P3. Any unspecified one of terminals 211, 212, etc., or the terminals 211, 212, etc. collectively, is also referred to as a terminal 21. Any unspecified one of the switching circuits 221, 222, etc., or the switching circuits 221, 222, etc. collectively, is also referred to as a switching circuit 22. When the switching circuit 22 is not mounted on the second mounts MUX1 to MUX3, the wiring lines on both sides of each of the second mounts MUX1 to MUX3 are not connected to each other. Regardless of whether the passive component 23 is mounted on the third mounts P2 and P3, the wiring lines on both sides of the third mounts P2 and P3 are connected. The first connection circuit 201 does not include a third mount. Each mount is typically provided with an indicator that makes it possible to identify which component is to be connected. That is, by checking the indicator, the user can identify the type of electronic component to be mounted. In addition, an instruction manual or other documentation (e.g., manuals, specifications, or circuit diagrams) for the circuit board 20 or the ultrasonic diagnostic apparatus 1 may include information regarding electronic components that are not currently mounted on respective mounts but may be mounted in the future, and information regarding electronic components that are to be removed when such additional mounting is performed.

When the maximum number connectable to the circuit board 20 is two, the terminal 211 is mounted on the first mount CN1, and the switching circuit 221 is mounted on the second mount MUX1. The terminal 212 may be mounted on the first mount CN2, and the terminal 21 need not be mounted on the first mount CN3. The switching circuit 222 may be mounted on the second mount MUX2, and a switching circuit 22 need not be mounted on the second mount MUX3. On the other hand, the passive component 23 need not be mounted on the third mount P2, and a passive component 233 may be mounted on the third mount P3. In the second connection circuit 202, a circuit system in which the terminal 21 and the switching circuit 22 are mounted and no passive component 23 is mounted is referred to as a first circuit.

The first mount CN1 and the second mount MUX1, the first mount CN2 and the second mount MUX2, and the first mount CN3 and the second mount MUX3 are each connected by the same number of wiring lines as the number of transducers. These wiring lines can be connected to respective signal lines of the cable 31 via the terminals 211 and 212. The number of wiring lines between each of the second mounts MUX1 to MUX3 and the connection terminal C2 may be, for example, one-half of the number of transducers. In this case, the signal is a two-phase signal, and signals for the two transducers are alternately supplied.

The passive component 233 mounted on the third mount P3 may include a capacitor. The other end of the passive component 233 is grounded to the housing. The wiring lines and a ground plane may be patterned on the circuit board 20. Instead of being mounted, the passive component 23 may be pre-patterned on the circuit board 20, and may be separated from the third mounts P2 and P3 when unnecessary.

The switching circuit 22 switches between the drive signal transmitted from the transmission driving section 12 to the ultrasound probe 30 and a reception signal transmitted from the ultrasound probe 30 to the reception driving section 13. The switching circuit 22 may be, for example, a multiplexer. In a case where a single multiplexer cannot be connected to all wiring lines, a plurality of multiplexers may be arranged in parallel. The switching circuit 22 switches whether a wiring line connection is established so that signals are selectively input to and output from the selected ultrasound probe 30.

Here, the first connection circuit 201 on which the terminal 211 and the switching circuit 221 are mounted, and the second connection circuit 202 on which the terminal 212 and the switching circuit 222 are mounted, each have an impedance corresponding to the drive frequency in transmitting and receiving signals. The second connection circuit 203 on which the passive component 233 is mounted, i.e., the first circuit, may have the same impedance as the first connection circuit 201 and the second connection circuit 202 at the drive frequency. As a result, the difference in impedance on the circuit board 20 is reduced between a case where the maximum number of mounts equals the maximum connectable number and a case where the maximum connectable number is less than the maximum number of mounts. That is, even when the number of the switching circuits 22 and the terminals 21 mounted on the same circuit board 20 is any number no more than the maximum number of mounts, the impedance can be readily maintained at substantially the same level. As used herein, “the same" does not mean "exactly the same". A deviation may be permitted within a range in which adverse effects on the operation are not significant and variations in passive components are allowable, for example, a deviation of about 10% to 20% may be permitted.

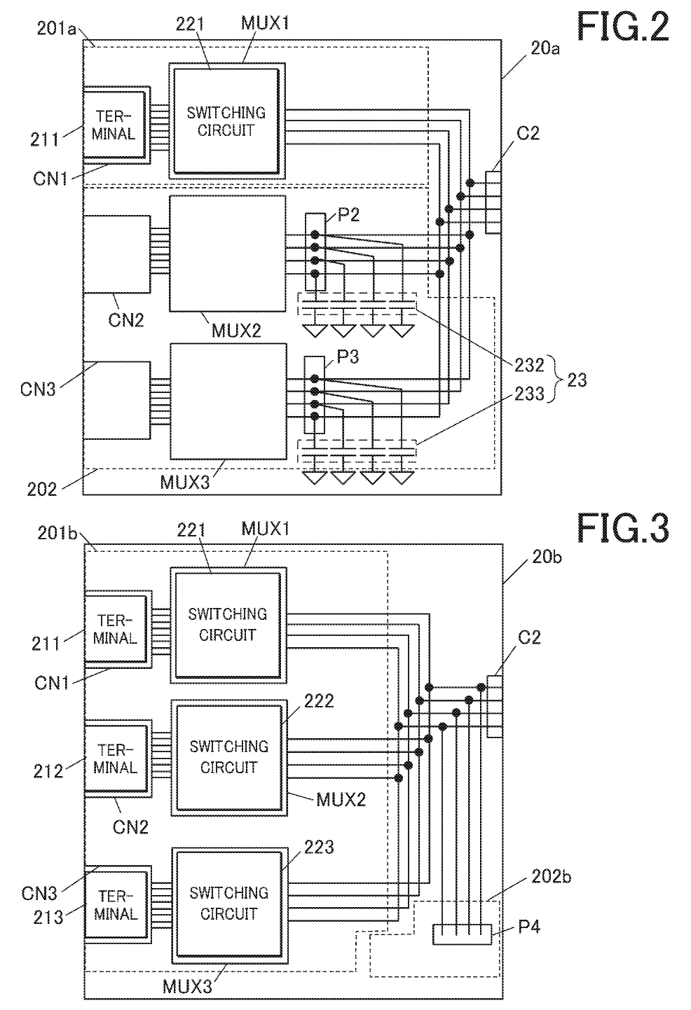

FIG. 2 is a diagram illustrating an example in which the single terminal 211 is located on a circuit board 20a.

That is, in one embodiment, the maximum number connectable to the circuit board 20a may be 1. When the single terminal 211 and the switching circuit 221 are located on the circuit board 20a, the terminal 211 and the switching circuit 221 are located on the first connection circuit 201. On the other hand, the switching circuit 22 and the terminal 21 are not located in the second connection circuits 202 and 203. In the plurality of second connection circuits 202, passive components 232 and 233 may be mounted on the third mounts P2 and P3, respectively.

FIGS. 3-5 are diagrams illustrating examples of other circuit boards 20b to 20d.

Unlike the above, the circuit boards 20b to 20d may include one second connection circuit 202b having a third mount P4, without having a first mount and a second mount. On the other hand, the circuit boards 20b to 20d include a plurality of first connection circuits 201b to 201d (e.g., three first connection circuits), each having a first mount and a second mount and not having a third mount.

FIG. 3 shows the circuit board 20b in a case where the maximum connectable number is equal to the maximum number of mounts.

The terminals 211 to 213 are mounted on the first mounts CN1 to CN3 of the first connection circuit 201b, respectively. The switching circuits 221 to 223 are mounted on the second mounts MUX1 to MUX3, respectively. On the other hand, no passive components are mounted on the third mount P4 of the second connection circuit 202b. That is, in this case, impedance adjustment using passive components is unnecessary.

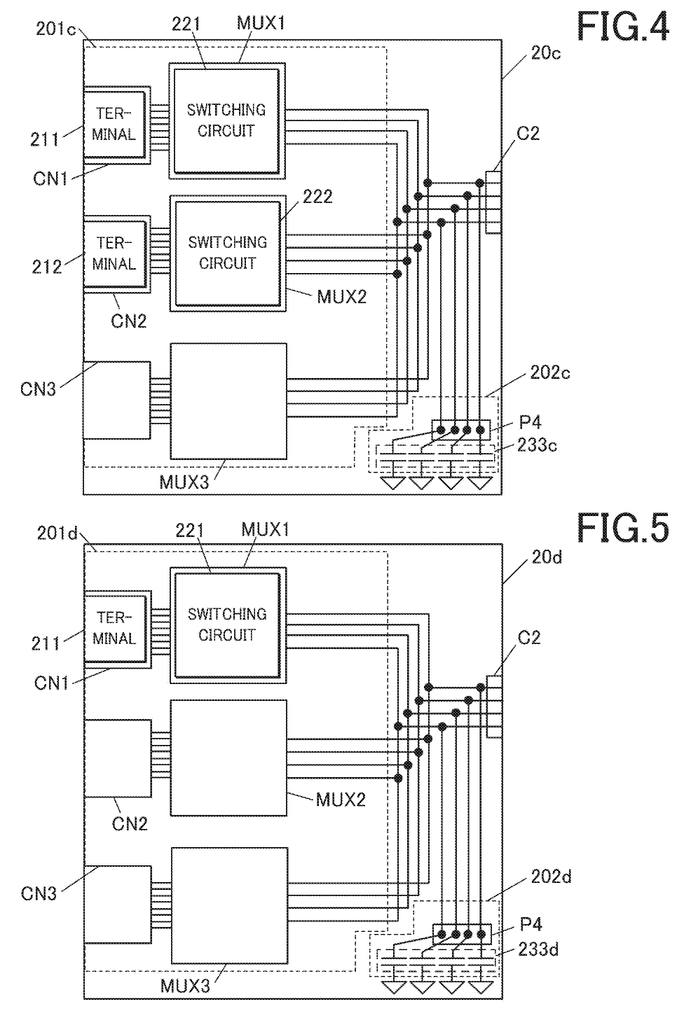

FIG. 4 shows the circuit board 20c in a case where the maximum connectable number is one less than the maximum number of mounts. In the circuit board 20c, in the first connection circuit 201c, the switching circuit is not mounted on the second mount MUX3, and the terminal is not mounted on the first mount CN3. On the other hand, the passive component 233c is mounted on the third mount P4 of the second connection circuit 202c. The impedance of a system in the second connection circuit 202c including the passive component 233c is equivalent to the impedance of the system in the first connection circuit 201c in which the terminal 211 and the switching circuit 221 are mounted. Accordingly, the overall impedance of the circuit board 20c becomes equal to that in a case where the maximum number of mounts of the terminals and switching circuits are mounted. The system of the first connection circuit 201c including the terminal 21 and the switching circuit 22 is referred to as a second circuit, and the system of the first connection circuit 201c in which the terminal 21 and the switching circuit 22 are not mounted is referred to as a third circuit. In the circuit board 20c of FIG. 4, the configuration is such that the switching circuit is not mounted on the second mount MUX3 and the terminal is not mounted on the first mount CN3, however, the present disclosure is not limited thereto. For example, a configuration may be adopted in which the switching circuit is not mounted on the second mount MUX3 and the terminal is mounted on the first mount CN3.

As shown in FIG. 5, when the maximum connectable number is less than the maximum number of mounts by at least two, at least two systems of third circuits in which the terminal 21 and the switching circuit 22 are not mounted are present in the first connection circuit 201d. In this case, in the second connection circuit 202d, a common passive component 233d may be mounted for the at least two systems of the third circuit. That is, the circuit board 20d includes a plurality of third circuits, and includes one second connection circuit 202d that has the third mount P4 on which the passive component 233d is mounted, and that has neither a first mount nor a second mount. In this case, the passive component 233d of the second connection circuit 202d may be, for example, the capacitor having a capacitance equal to a sum of capacitances of the capacitor that is the passive component 232 included in the second connection circuit 202 and the capacitor that is the passive component 233 included in the second connection circuit 203, respectively, in FIG. 2. In other words, the capacitance of the passive component 233d is a capacitance value obtained by multiplying, by an integer, the impedance of the second circuit on which the terminal 211 and the switching circuit 221 are mounted. The integer is determined by the number of third circuits. Thus, the number of passive components can be reduced in inverse proportion to the number of third circuits. In the circuit board 20d of FIG. 5, two sets of terminals 21 and switching circuits 22 are not mounted, however, the present disclosure is not limited thereto. For example, the circuit board 20d may be configured such that two sets of switching circuits 22 are not mounted and corresponding terminals 21 are mounted.

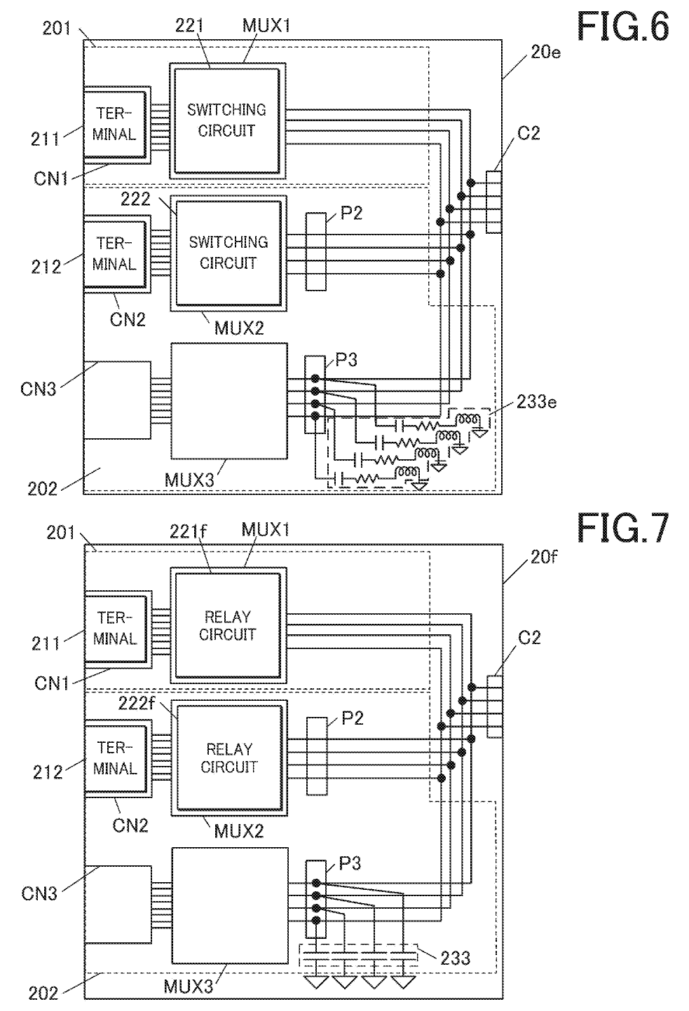

FIG. 6 is a view showing another example of the circuit board 20e.

The wiring lines and the mounts of the circuit board 20e are the same as those of the circuit board 20 shown in FIG. 2. In one embodiment, a passive component 233e is not limited to the capacitor. The passive component 233e may include a resistance element and/or an inductor. These may be used, in particular, for fine adjustment of the impedance using the capacitor. Here, the resistance element and the inductor are connected in series with the capacitor, however, one or both of them may be connected in parallel with the capacitor.

FIG. 7 is a diagram illustrating another example of the circuit board 20f.

The wiring lines and the mounts of the circuit board 20f are the same as those of the circuit board 20 shown in FIG. 2. According to one embodiment, relay circuits 221f and 222f may be used as the switching circuit 22 instead of the multiplexer.

As described above, the circuit board 20 according to the present embodiment includes the connection terminal C2 connected to the main body 10 of the ultrasonic diagnostic apparatus 1, the first connection circuit 201, and the second connection circuit 202. The first connection circuit 201 includes the first mounts CN1 and CN2 and the second mounts MUX1 and MUX2. The first mounts CN1 and CN2 each have one end at the connection terminal C2 and are configured to mount the terminal 21 used to connect the ultrasound probe 30. The second mounts MUX1 and MUX2 are located between the first mounts CN1 and CN2 and the connection terminal C2. The second mounts MUX1 and MUX2 are configured to mount the switching circuit 22. The second connection circuit 202 includes the third mount P3. The third mount P3 has one end at the connection terminal C2 and is configured to mount the passive component 23. At least one of the first connection circuits 201 includes the terminal 211 mounted on the first mount CN1 and the switching circuit 221 mounted on the second mount MUX1. There are at least two sets each including the first mount and the second mount. The circuit board 20 is in one of the following states. (1) The terminal is mounted on all of the first mounts, the switching circuit is mounted on all of the second mounts, and no passive components are mounted on the third mount. (2) The switching circuit is not mounted on some of the second mounts, and the passive component is mounted on at least some of the third mounts. As described above, when the circuit board 20 has mounting space for the terminals 21 and the switching circuits 22 required to enable a plurality of ultrasound probes 30 to be connected to the circuit board 20, it is not necessary to mount the terminal 21 and the switching circuit 22 in some of the mounting spaces. Accordingly, the number of terminals 21 can be flexibly set to an appropriate number in accordance with the user's request in the common circuit board 20. On the other hand, simply changing the number of terminals 21 and the number of switching circuits 22, particularly reducing the number of switching circuits 22, changes the impedance of the circuit board 20. Therefore, by including the passive component 23 to compensate for the absence of the switching circuits 22 and the like, a change in impedance in accordance with the number of terminals 21 and switching circuits 22 is reduced in the circuit board 20. Therefore, in the ultrasonic diagnostic apparatus 1 using the circuit board 20, cost is reduced by the common circuit board 20 while a decrease in image quality due to a change in the number of switching circuits 22 is reduced.

Further, the second connection circuit 202 may include the first mount CN3 and the second mount MUX3. In the second connection circuit 202, the terminal 213 may be mounted on the first mount CN3, the switching circuit 223 may be mounted on the second mount MUX3, and no passive component may be mounted on the third mount P3. Alternatively, in the second connection circuit 202, the switching circuit 22 may be absent from the second mount MUX3, and the passive component 233 may be mounted on the third mount P3. That is, the second connection circuit 202 may include the first mount CN3, the second mount MUX3, and the third mount P3. In this case, by selectively mounting either the terminal 21 and the switching circuit 22 or the passive component 233, a change in impedance of the second connection circuit 202 is reduced. Thus, the circuit board 20 can easily maintain the impedance of each connection circuit.

The circuit board 20 may include a plurality of second connection circuits 202. The second connection circuits 202 may include at least one first circuit in which the passive component 23 is mounted on the third mounts P2 and P3. That is, when the maximum connectable number is smaller than the maximum number of mounts, the impedance of each second connection circuit 202 on which the passive component 23 is mounted may be substantially equal to the impedance of the first connection circuit 201 at the drive frequency of the ultrasound probe 30. By mounting the passive component 23 so as to be appropriately matched to the impedance of the first connection circuit 201, the circuit board 20 can effectively reduce an adverse effect on the signal.

Alternatively, each of the circuit boards 20b to 20d may include a plurality of first connection circuits 201b to 201d. On the other hand, each of the circuit boards 20b to 20d may include a single second connection circuit 202b to 202d. Each second connection circuit 202b to 202d need not include the first mount and the second mount. As in the circuit board 20b, the terminal 21 may be mounted on all of the first mounts of the first connection circuit 201b, the switching circuit 22 may be mounted on all of the second mounts, and the passive component 23 need not be mounted on the third mount P4 of the second connection circuit 202b. Alternatively, as in the circuit boards 20c and 20d, the switching circuit 22 need not be mounted on the second mounts in some of the first connection circuits 201c and 201d. In this case, the passive components 233c and 233d are mounted on the third mount P4 of the second connection circuits 202c and 202d. That is, regardless of the number of the difference between the maximum number of mounts and the maximum connectable number, the single second connection circuit 202b to 202d may adjust, using the passive component 23, the impedance according to the difference. Accordingly, the third mount P4 can be fixed as a single unit, thereby reducing space. Further, even when the maximum connectable number is small, the number of passive components 23 mounted on the third mount P4 does not increase, thereby minimizing the labor required for mounting.

Further, the plurality of first connection circuits 201c and 201d may include the second circuit and the third circuit. In the second circuit, the terminal 21 is mounted on the first mount, and the switching circuit 22 is mounted on the second mount. In the third circuit, the switching circuit 22 is not mounted on the second mount. In this case, the passive components 233c and 233d are mounted on the third mount P3 of the second connection circuits 202c and 202d. At the drive frequency of the ultrasound probe 30, the impedance of the second connection circuit 202c and the impedance of the second connection circuit 202d may be integer multiples of the impedance of the second circuit. That is, the second connection circuits 202c and 202d may collectively have the impedance of at least one third circuit. Thus, the circuit that does not include the passive component 23 can be included, and the number of passive components 23 can be reduced.

In particular, the impedance of the second connection circuit 202 may be determined in accordance with the number of third circuits. That is, the passive components 233c or 233d of the single second connection circuit 202c or 202d may collectively bear all of the impedance corresponding to the number, relative to the maximum number of mounts, of the terminals 21 and the switching circuits 22 that are not mounted. As a result, the number of passive components 23 can be reduced to a minimum, thereby effectively reducing the cost and labor associated with mounting the passive components 23.

Further, the switching circuit 22 may be the multiplexer. Thus, a large number of wiring lines can be switched easily and reliably.

Alternatively, the switching circuits 22 may be relay circuits 221f and 222f. This also facilitates switching of a large number of wiring lines.

The passive component 23 may include the capacitor. One end of the capacitor may be grounded. The capacitor is a suitable passive component because it consumes essentially no power and effectively functions as the impedance for AC signals.

The passive component 233e may further include at least one of the resistance element and the inductor. Compared with the capacitor, the addition of the resistance element and/or the inductor facilitates fine adjustment of the impedance.

The ultrasonic diagnostic apparatus 1 according to the present embodiment includes any of the circuit boards 20 to 20e and the main body 10. The ultrasonic diagnostic apparatus 1 can include the terminals 21 for connecting an appropriate number of ultrasound probes 30 desired by the user while reducing development and manufacturing costs by using the common circuit board 20. Accordingly, the ultrasonic diagnostic apparatus 1 can be provided with greater flexibility while preventing increases in cost and labor.

An object of the present disclosure is to provide a circuit board and an ultrasonic diagnostic apparatus capable of connecting an appropriate number of ultrasound probes while suppressing a decrease in measurement accuracy.

The present disclosure is merely illustrative, and various modifications can be made.

For example, in the above description, branch lines branched from the wiring line connected to the switching circuit 22 of the first connection circuit 201 are illustrated as the second connection circuits 202 and 203, however, the present disclosure is not limited thereto. The position at which one or more second connection circuits 202 branch between the connection terminal C2 and the second mount MUX1 may be set as appropriate, either individually or in common.

In addition, the impedance of the passive component 23 need not be accurately matched to the impedance of the first connection circuit 201. If the impedance of the second connection circuit 202 and the like becomes closer, even to some extent, to the impedance of the first connection circuit 201, adverse effects on the reception signal and image quality of the generated image are reduced.

In the above description, the multiplexer and the relay circuit are exemplified as the switching circuit 22, however, the switching circuit 22 is not limited thereto. Another switching circuit may be used, as long as it can appropriately control switching of the connection destination of the signal line.

In addition, the positional relationship among the passive components 23 may be arbitrarily determined. In addition, the passive components 23 need not include, for each wiring line, one element of the same type, for example, one capacitor. A plurality of passive components 23 of the same type may be arranged in series and/or in parallel. In addition, the plurality of passive components 23 connected to the same wiring line may be arbitrarily connected in series and/or in parallel. Further, the circuit board 20 may be any type of board, such as a double-sided board, a multilayer board, a laminated board, or a flexible board.

In addition, circuit parameters of the passive components 23 connected to the respective wiring lines need not necessarily be the same. For example, the circuit parameters of the passive components 23 connected to the respective wiring lines may be adjusted, respectively, according to variations in the passive components 23 and/or circuit characteristics.

In the above description, an example has been described in which, when the maximum number of mounts is three, the maximum connectable number, i.e., the number of terminals 21, is one or two. However, the maximum number of mounts may be two, or may be four or more. When the maximum number of mounts is four or more, the number of common second connection circuits 202b to 202d may be at least two and less than the maximum number of mounts minus 1. Further, the circuit board 20 may include one second connection circuit having the first mount through the third mount. In this case, the common passive component may be mounted on the third mount of the second connection circuit depending on whether components are mounted on the first mount and the second mount of the second connection circuit and on whether components are mounted on the first mount and the second mount of another first connection circuit.

The main body 10 of the ultrasonic diagnostic apparatus 1 is not limited to the configuration described in the above embodiment. The main body 10 may include other configurations, and may be divided into a plurality of devices. For example, processing by the image generation section 15 and the image processing section 16 may be delegated to another device that is externally attached or connected to the network.

In addition, the specific configurations, contents and procedures of processing operations, and the like described in the above embodiments may be appropriately modified without departing from the scope of the present invention. The scope of the present invention encompasses the scope of the invention as set forth in the claims and equivalents thereof.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A circuit board comprising:
a connector to be connected to a main body of an ultrasonic diagnostic apparatus;
a first connection circuit including a first mount which includes one end at the connector, and on which a connection terminal used for connection to an ultrasound probe is to be mounted, and a second mount located between the first mount and the connector and on which a switching circuit is to be mounted; and
a second connection circuit including a third mount which includes one end at the connector, and on which a passive component is to be mounted,
wherein,
in at least one first connection circuit, the connection terminal is mounted on the first mount, and the switching circuit is mounted on the second mount,
at least two sets of the first mount and the second mount are provided, and
either the connection terminal is mounted on all of the first mounts, the switching circuit is mounted on all of the second mounts, and the passive component is not mounted on any third mount, or the switching circuit is not mounted on at least one of the second mounts, and the passive component is mounted on at least one of the third mount.

2. The circuit board according to claim 1, wherein the second connection circuit includes the first mount and the second mount, and either, the connection terminal is mounted on the first mount of the second connection circuit, a switching circuit is mounted on the second mount of the second connection circuit, and the passive component is not mounted on the third mount, or the switching circuit is not mounted on the second mount, and the passive component is mounted on the third mount.

3. The circuit board according to claim 2,
further comprising a plurality of second connection circuits,
wherein,
the second connection circuit includes at least one first circuit in which the passive component is mounted on the third mount, and
at a drive frequency of the ultrasound probe, an impedance of the first circuit is equal to the impedance of the first connection circuit.

4. The circuit board according to claim 1,
further comprising a plurality of first connection circuits, and
one second connection circuit,
wherein,
the second connection circuit does not include the first mount and the second mount, and
the connection terminal is mounted on all first mounts of the first connection circuits, the switching circuit is mounted on all second mounts, and the passive component is not mounted on the third mount of the second connection circuit, or in at least one of the first connection circuits, the switching circuit is not mounted on the second mount, and the passive component is mounted on the third mount of the second connection circuit.

5. The circuit board according to claim 4, wherein,
the plurality of first connection circuits include, a second circuit in which the connection terminal is mounted on the first mount and the switching circuit is mounted on the second mount, and a third circuit in which the switching circuit is not mounted on the second mount, and the passive component is mounted on the third mount of the second connection circuit, and
at a drive frequency of the ultrasound probe, an impedance of the second connection circuit is an integer multiple of the impedance of the second circuit.

6. The circuit board according to claim 5, wherein the impedance of the second connection circuit is determined in accordance with a number of the third circuits.

7. The circuit board according to claim 1, wherein the switching circuit is a multiplexer.

8. The circuit board according to claim 1, wherein the switching circuit is a relay circuit.

9. The circuit board according to claim 1, wherein,
the passive component includes a capacitor, and
one end of the capacitor is grounded.

10. The circuit board according to claim 9, wherein the passive component includes at least one of a resistance element and an inductor.

11. An ultrasonic diagnostic apparatus comprising:
a circuit board according to claim 1; and
a main body of an ultrasonic diagnostic apparatus.
